(19) United States
(12) Patent Application Publication
(10) Pub. No.: US 2023/0057906 A1
(43) Pub. Date: Feb. 23, 2023

(54) MULTIPLE COMPONENT GRAFTS FOR TREATING TISSUE DEFECTS AND METHODS FOR MAKING AND USING SAME

(71) Applicant: Musculoskeletal Transplant Foundation, Edison, NJ (US)

(72) Inventors: Marc Long, Denville, NJ (US); Eric J. Semler, Morganville, NJ (US); Mat Citro, Matawan, NJ (US); Evangelia Chnari, Scotch Plains, NJ (US)

(21) Appl. No.: 17/792,546

(22) PCT Filed: Jan. 14, 2021
(86) PCT No.: PCT/US2021/013464

§ 371 Date: Jul. 13, 2022

Related U.S. Application Data
(60) Provisional application No. 62/961,138, filed on Jan. 14, 2020.

(51) Int. Cl. A61L 27/36

(52) U.S. Cl. CPC A61L 27/3604; A61L 27/3645

(57) ABSTRACT

Multiple component grafts are provided for treatment of tissue defects and comprise two or more components, each of which is a tissue-derived matrix and at least two of which are derived from different types of tissue. For example, a first component may be a matrix derived from cartilage tissue such as cartilage fibers with or without viable cells, cartilage particles with or without viable cells, or combinations of any two or more such cartilage-derived matrices. A second component may be a matrix derived from bone tissue such as mineralized or demineralized cortical bone fibers, viable cancellous bone matrix (e.g., cryopreserved or lyophilized chips, particulates, powder, sheets, putty, flowable fluid, etc.), demineralized or demineralized cancellous bone matrix (chips, particulates, powder, sheets, putty, flowable fluid, etc.), or combinations of any two or more of such bone-derived matrices. Also provided are methods for making and using such multiple component grafts.

### CROSS-REFERENCE TO RELATED APPLICATIONS

The present application is the national stage of International Patent Application No.: PCT/US2021/013464, filed Jan. 14, 2021, which claims the benefit of U.S. Provisional Application No. 62/961,138, filed on Jan. 14, 2020, the entire disclosure of which is hereby incorporated by reference herein.

### FIELD OF THE INVENTION

The present invention relates generally to multiple component grafts for treating tissue defects, as well as methods for making and using such grafts. More particularly, the present invention relates to grafts which comprise two or more components, each of which comprises a tissue-derived matrix, at least two of which are derived from different types of tissue.

### BACKGROUND

A live subject may sustain damage or injury to one or more organs, tissues, or portions or combinations thereof, from any number of causes including, but not limited to, trauma, disease, atrophy, surgery, other therapeutic or cosmetic procedure, etc., resulting in a tissue defect. Tissue defects may be external, internal, or both, and are often responsive to treatment using a graft comprising one or more tissue-derived matrices. Typical treatments involve applying or implanting one or more such tissue-derived matrices, or a graft comprising them, proximate (i.e., in, on, adjacent, or a combination thereof) to the tissue defect. Some tissue defects involve or include two of more types of tissue.

Tissue-derived matrices are generally produced from tissue samples recovered from one or more donors (living or deceased) and may be autogenic, allogenic, xenogenic, or combinations thereof. Many different types of tissue samples are known and used to produce tissue-derived matrices. Moreover, the tissue-derived matrices and grafts containing one or more of them are produced and available in any of several different physical forms including, without limitation, pieces, particles, fibers, powder, sheets, putty, flowable fluid, three-dimensional shapes which are monolithic, multi-piece, or otherwise formed (e.g., molded or otherwise shaped) from particles, fibers, pieces, as well as combinations of any of the foregoing physical forms. Many such tissue-derived matrices and grafts comprising them have been shown to enable, facilitate, and/or enhance one or more of tissue healing, repair, and regeneration at the tissue defect.

Tissue defects are often, but not always, treated with a graft comprising one or more matrices derived from the same type of tissue as is present at or proximate to the tissue defect to be treated. For example, defects in bone have been treated by implanting a graft comprising one or more bone-derived matrices, including cancellous or cortical bone (or both), demineralized or not, viable or not, and in any one or more of various physical forms. Similarly, defects in cartilage have been treated by implanting a graft comprising one or more cartilage-derived matrices, whether viable or not, and in any one or more of various physical forms.

On the other hand, tissue defects involving or including primarily one type of tissue have also been successfully treated by implanting grafts comprising one or more tissue-derived matrices produced from a tissue type different than the one tissue type present at the defect to be treated. For example, without limitation, grafts comprising one or more placenta derived matrices (e.g., an amnion derived matrix, a chorion derived matrix, or both, etc.) have been used, for example without limitation, to treat defects in bone, cartilage, eyes, dermis, cardiac and other tissue types.

In some cases, a defect may involve more than one type of tissue. For example, damage, injury, or surgery to a joint may create a tissue defect which involves both bone and cartilage tissues. Similarly, damage or injury to the face of a subject may create a tissue defect which includes two or more of ocular, skin, muscle, and bone tissues. While cartilage tissue (e.g., fibers, particles, sponges, putties, etc.) has been used clinically in human clinical indication for repair of chondral defects, and while bone tissues have been used clinically in human clinical indications to repair bony defects, use of grafts comprising combinations of cartilage-derived tissue forms and bone-derived tissue forms have only started to be explored for the treatment of an osteochondral defect.

Such osteochondral defects are currently often treated with either monolithic autologous or allogeneic osteochondral plugs. Instrumentation is typically used to prepare the defect so that a pre-shaped implant can implanted in a press-fit fashion and the curvature of the joint surface can be appropriately restored. This approach is limited to natural anatomical configurations of osteochondral tissue and may also involve removing healthy joint tissue to better accommodate the shape of the pre-shaped implant.

It may be advantageous to implant, in or proximate to such defects, grafts comprising two or more components, each of which comprises a tissue-derived matrix and at least two of which are derived from different types of tissue, and where each component is implanted in or proximate to the tissue type in the defect to be treated by each respective component. The present invention provides such multiple component grafts, methods for producing them, and methods for their use to treat such multiple tissue defects with a multiple component graft which comprises two or more components, each of which comprises a tissue-derived matrix and at least two of the tissue-derived matrices are derived from different types of tissue samples.

### SUMMARY OF THE INVENTION

The present invention provides a multiple component graft for treating a tissue defect, comprising two or more components, each of which comprises a tissue-derived matrix or a multiple tissue matrix, wherein at least two of the components are derived from and comprise different types of tissue. The types of tissue are selected from: adipose, amnion, amniochorion, artery, bone, cartilage, chorion, colon, dental, dermal, duodenal, endothelial, epithelial, fascial, gastrointestinal, growth plate, intervertebral disc, intestinal mucosa, intestinal serosa, ligament, liver, lung, mammary, meniscal, muscle, nerve, ovarian, parenchymal organ, pericardial, periosteal, peritoneal, skin, spleen, stomach, synovial, tendon, testes, umbilical cord, urological, vascular, vein, Whartons jelly, and combinations thereof. Each tissue-derived matrix and multiple tissue matrix, independently, may have a physical form selected from: pieces, particles, fibers, powder, sheets, putty, flowable fluid, three-dimensional shapes, and combinations thereof. The three-dimensional shapes may be selected from: monolithic, multi-piece, shapes otherwise formed, molded or shaped from other physical forms, and combinations thereof

In an exemplary embodiment, the multiple component graft comprises a first component which comprises a first tissue-derived matrix or a first multiple tissue matrix which includes at least a first type of tissue and at least one physical form, and a second component which comprises a second tissue-derived matrix or a second multiple tissue matrix which includes at least a second type of tissue which is different from the first type of tissue and at least one physical form. For example, the first component may comprise a bone-derived matrix, the first type of tissue may be, and the first physical form may be selected from pieces, granules, particles, powder, fibers, putty, and a combination thereof, and the second component may comprise a cartilage-derived matrix, the second type of tissue may be cartilage, and the second physical form may be selected from pieces, granules, particles, powder, fibers, putty, and a combination thereof.

The present invention also provides a method of producing the multiple component graft of claim 1, comprising the steps of: providing the two or more components, and sequentially implanting each of the two or more components, one at a time, proximate the tissue defect. In an exemplary embodiment, the step of providing the two or more components is performed by obtaining: a first tissue-derived matrix or a first multiple tissue matrix which includes at least a first type of tissue and at least one physical form, and a second component which comprises a second tissue-derived matrix or a second multiple tissue matrix which includes at least a second type of tissue which is different from the first type of tissue and at least one physical form, and wherein the step of sequentially implanting is performed by implanting each of the two or more components, one at a time, proximate the tissue defect, wherein either the of the first or second components is implanted first.

In an exemplary embodiment, the tissue defect is an osteochondral defect in a joint having a bone portion and a cartilage portion, where the bone portion is located deeper in the defect, wherein the first component of the multiple component graft comprises a bone-derived matrix and the first type of tissue is bone, the second component of the multiple component graft comprises a cartilage-derived matrix and the second type of tissue is cartilage, the at least one physical form of each of the bone-derived matrix and cartilage-derived matrix is, independently, selected from pieces, granules, particles, powder, fibers, putty, and a combination thereof, and wherein the step of sequentially implanting is performed by first implanting the bone-derived component proximate the bone portion of the osteochondral defect, and thereafter, implanting the cartilage-derived matrix proximate the bone-derived matrix and the cartilage portion of the osteochondral defect.

The present invention also provides a method of producing the multiple component graft, comprising the steps of: providing the two or more components, and prior to implanting the two or more components, combining two or more of the two or more components together to form one or more composite components and optionally, leaving one or more of the two or more components as one or more uncombined components, and sequentially implanting each of the one or more composite components and, if present, each of the one or more uncombined components, one at a time, proximate the tissue defect.

The present invention also provides a method for treating an external or internal tissue defect of a subject using the multiple component graft, comprising the steps of: optionally, preparing the tissue defect to receive the multiple component graft; providing the multiple component graft, already assembled or not; and implanting the multiple component graft proximate the tissue defect according to either of the above methods of producing the multiple component graft.

### DETAILED DESCRIPTION

Detailed embodiments of the present invention are disclosed herein. It should be understood that the disclosed embodiments are merely illustrative of the invention which may be embodied in various forms. In addition, each of the examples given in connection with the various embodiments of the invention is intended to be illustrative, and not restrictive. Therefore, specific structural and functional details disclosed herein are not to be interpreted as limiting, but merely as examples for teaching one skilled in the art to variously employ the present invention.

As used herein, the term “biomaterial” is used to mean a material or matrix derived in whole or in part from tissue recovered from a donor which is the same individual (autogenic), different individual(s) but same species (allogenic), or different species (xenogenic), as the individual being treated with the biomaterial.

As used herein, the terms “implant” and “graft” are used interchangeably, unless indicated otherwise.

The present invention provides multiple component grafts which comprise two or more components, each of which comprises a tissue-derived matrix (or a multiple tissue matrix) and at least two of which are derived from different types of tissue. Methods of using the multiple component graft comprise implanting (i.e., applying, placing, inserting, etc.) the graft proximate (in, on, or both) to a tissue defect in a subject so that each respective component is positioned proximate to or in contact with that portion of the defect comprising the tissue type to be treated by each respective component. The tissue defect may, but is not required to, involve or have at least two different tissue types proximate thereto. The multiple component tissue graft may, for example without limitation, be formed or assembled from the two or more components prior to implanting (i.e., the two or more components are assembled together prior to implanting proximate the tissue defect), or during implanting (i.e., at least two of the two or more components are sequentially implanted proximate the tissue defect and one another), wherein each component comprises a tissue-derived matrix or a multiple tissue matrix. The components of a multiple component graft are not mixed or blended together in a substantial fashion, but rather, are assembled, constructed, interlocked, attached, layered, etc. As used herein in connection with formation of the multiple component graft using two or more components, the term “assembled” includes any one or more of: constructed, oriented, placed, combined, layered, stacked, connected, bound, affixed, etc., but not substantially mixed or blended (i.e., any inadvertent or consequential mixing or interspersing of adjacent components or matrices comprising them is limited to occurring proximate to the interface where they contact one another).

As mentioned above, according to the invention described and contemplated herein, the multiple component graft comprises at least a first component and a second component which are assembled together, either before or during implanting proximate a tissue defect. The designation of “first,” “second,” “third,” etc., regarding each component of any embodiment of the multiple component tissue graft is not intended to, and does not, indicate or require any particular order of assembly or implantation, but rather is intended only to be useful in distinguishing between the two or more components of any particular multiple component graft. For example, a multiple component graft which comprises first, second, and third components does not have to be assembled with the first, second and third components in sequential order according to those designations. Rather, when such a multiple component graft is assembled prior to implantation, for example without limitation, the third component may be positioned and assembled in between the second and first components, or the first component may be positioned between the second and third components. Similarly, when such a multiple component graft is assembled during implanting proximate a tissue defect, the components may be implanted in any order, i.e., with any of the third, second, or first being implanted first, and any of the first, second, or third components being implanted last.

“Tissue-derived matrices” are generally produced from tissue samples of a single tissue type which are recovered from one or more donors (living or deceased) and may be autogenic, allogenic, xenogenic, or combinations thereof. Accordingly, as used herein, each “tissue-derived matrix” is derived from a single tissue type. Each tissue derived matrix may, independently of others, have any of several possible physical forms including, without limitation, pieces, particles, fibers, powder, sheets, putty, flowable fluid, three-dimensional shapes which are monolithic, multi-piece, or otherwise formed (e.g., molded or otherwise shaped) from particles, fibers, pieces, as well as combinations of any of the foregoing physical forms. For example, without limitation, cartilage-derived matrix may include 100% cartilage fibers, or 100% cartilage particles, or 50% cartilage fibers and 50% cartilage particles, or any proportion of cartilage fibers and particles desired between 1%:99% and 99%:1%. Furthermore, a tissue-derived matrix may be derived from and comprise only one of autogenic, allogenic, or xenogenic tissue samples of the same tissue type.

Furthermore, a “tissue-derived matrix” may be derived from and comprise two or more of autogenic, allogenic, and xenogenic tissue samples, where all of the tissue samples are the same tissue type, but from different kinds of donors. For example, without limitation, a tissue-derived matrix comprising cartilage tissue (i.e., a cartilage-derived matrix) may be derived from and comprise cartilage samples recovered from a deceased human donor (allogenic) as well as cartilage samples recovered from the recipient subject (autogenic) whose tissue defect is being treated using a multiple component graft which comprises the cartilage-derived matrix and another tissue-derived matrix comprising another tissue type (e.g., bone, amnion, etc.).

A matrix which is derived from, and therefore comprises, two or more tissue types will be referred to hereinafter as a “multiple tissue matrix.” Accordingly, for example, a matrix derived from and containing both cortical bone and cancellous bone would be referred to as a multiple tissue matrix or cortical and cancellous bone-derived matrix. Similarly, a matrix derived from and containing both amnion and chorion tissue would be referred to as a multiple tissue matrix or an amnion and chorion-derived matrix. In multiple tissue matrices, the different tissue types may have the same or different physical forms and may or may not be homogenous with regard to the different tissue types or different physical forms. Thus, unlike the components of the multiple component graft, it is contemplated that the different tissue types and physical forms thereof in a multiple tissue matrix may be mixed, blended, homogenized, etc., together, as well as assembled, constructed, layered, etc. As will be readily understood by persons of ordinary skill in the relevant art, it is contemplated and possible that where two or more tissue-derived matrices are combined to form a multiple tissue type by mixing, blending, etc., the resulting multiple tissue matrix may be homogenous or not. In some embodiments, such a multiple tissue matrix may, intentionally or unintentionally, have gradients of different concentrations of the different tissue-derived matrices combined to form the multiple tissue matrix.

Furthermore, the different tissue types included in a multiple tissue matrix may also, independently, each be a combination of autogenic, allogenic, and xenogenic tissue samples. Since a multiple component graft comprises two or more components, when a multiple component graft comprises a first component which comprises a multiple tissue matrix as described above, the graft also necessarily comprises at least a second, separate component. Furthermore, the second component will comprise either a tissue-derived matrix or a multiple tissue matrix which is at least partly derived from a tissue type which is different from at least one of the tissue types included in the multiple tissue matrix of the first component.

In some embodiments, a multiple tissue matrix may be formed by combining tissues samples of different tissue types at some point during a processing method which produces the multiple tissue matrix. For example, a bone tissue sample may be partially processed (cut into pieces, cleaned, disinfected, demineralized, and milled into granules) and a cartilage tissue sample may also be partially processed (cut into pieces, cleaned, disinfected and milled into particles) separately from the bone tissue sample, and then the bone granules and cartilage particles mixed and lyophilized together to form a multiple tissue matrix comprising a mixture of lyophilized bone granules and cartilage particles. As another example, a bone tissue is partially processed (cut into pieces, milled into granules, and disinfected) to produce viable bone granules, and a cartilage tissue sample is partially processed (cleaned and shaved or grated to form cartilage fibers which are then disinfected) to produce viable cartilage fibers, followed by combining the bone granules and cartilage fibers and contacting them with one or more protectants before lyophilizing the mixture to form a multiple tissue matrix comprising a mixture of viable bone granules and viable cartilage fibers. Such a multiple tissue matrix as described above may be useful as one component of a multiple component graft, as described and contemplated herein, and assembled with another one or more components to form the multiple component tissue graft.

In other embodiments, a multiple tissue matrix may be formed by combining two or more tissue-derived matrices, each of which comprises a single tissue type processed entirely separately from other tissue types before combining to produce the multiple tissue matrix. For example, a bone tissue sample may be processed (cut into pieces, cleaned, disinfected, demineralized, lyophilized and milled into non-viable bone granules) to produce a bone-derived matrix comprising lyophilized bone granules, and a cartilage tissue sample may be processed (cleaned and shaved or grated to form cartilage fibers which are then disinfected and cryopreserved) to produce a cartilage-derived matrix comprising viable cartilage fibers, followed by combining the non-viable lyophilized bone granules and viable cartilage fibers together to form a multiple tissue matrix comprising a mixture of lyophilized bone granules and viable cartilage fibers. Such a multiple tissue matrix as described above may be useful as one component of a multiple component graft, as described and contemplated herein, and assembled with another one or more components to form the multiple component tissue graft.

Furthermore, each tissue-derived matrix and multiple tissue matrix may, independently of others, further include one or more additional materials such as: synthetic materials (e.g., ceramic, polymer, synthetic bone material, etc.), other exogenous materials (e.g., tri-calcium phosphate, hydroxyapatite, bioactive glass, porous metals such as titanium, tantalum, etc.), or extracellular matrix biomaterials, such as collagen, or natural- or animal-derived biomaterials, such as chitosan or alginate, bone marrow, bone marrow concentrate, blood-derived products, platelet-rich plasma (PRP), platelet-poor plasma (PPP), exogenous cells, or combinations thereof. It is noted that the exogenous cells may or may not be the same type of cells as are endogenous to the tissue types included in one or more of the components of the multiple component grafts and, separately, may or may not be autogenic, allogenic or xenogenic to the recipient and/or aforesaid tissue types. Likewise, each multiple component graft may further include one or more such additional materials. Additionally, one or more additional materials may be added to or combined with a tissue-derived matrix, a multiple tissue matrix or a multiple component graft at any point during formation, applying, or implanting them proximate to a tissue defect to be treated. In some embodiments, the additional materials include agents which are UV curable or cross-linkable so that one or more components, or even all components, of the multiple component graft is UV curable or cross-linkable to provide additional stability to the graft.

In some embodiments, the multiple component graft may be formed by sequentially implanting (i.e., applying, placing, inserting, etc.) each of the two or more tissue-derived matrices or multiple tissue matrices proximate (in, on, or both) to a tissue defect in a subject. In other embodiments, the multiple component grafts may be formed by assembling (constructing, orienting, placing, combining, layering, stacking, connecting, binding, affixing, etc., but not substantially mixing or blending) two or more matrices selected from tissue-derived matrices and multiple tissue matrices, together to form the multiple component graft prior to applying or implanting the graft proximate to a tissue defect. Furthermore, in some embodiments, two or more of the tissue-derived matrices may be assembled, combined, connected, layered, etc., together to form a multiple tissue matrix prior to sequentially applying or implanting the multiple component matrix, with or without one or more other tissue-derived matrices, proximate a tissue defect.

Additionally, in some embodiments, two or more matrices selected from a tissue-derived matrix and a multiple tissue matrix may be produced prior to sequentially applying or implanting them, with or without one or more additional matrices, proximate to a tissue defect. Tissue-derived matrices and multiple tissue matrices may be applied or implanted sequentially, in any order desired, proximate to a tissue defect to be treated.

In some embodiments, it is possible that a tissue-derived matrix may comprise a combination or mixture of tissue-derived biomaterials and be applied or implanted in or on a defect, prior to and/or after application or implanting of one or more single tissue-type matrices. Furthermore, each tissue-derived matrix and/or each multiple tissue matrix may be provided in a separate syringe or cannula for ease of delivery (application or implantation). In some embodiments, a series of tissue-derived matrices, each comprising different types of tissue, may be sequentially “loaded” in the syringe barrel for delivery proximate to a defect. Furthermore, in some embodiments, a series of one or more tissue-derived matrices and one or more multiple tissue matrices, may be sequentially “loaded” in the syringe barrel for delivery proximate to a defect.

Another possible method for producing or forming the multiple component grafts or portions thereof (e.g., one or more matrices, components, or portions thereof) is by 3-D printing. Any 3-D printing methods known now or in the future to persons of ordinary skill in the relevant art are suitable and practicable.

While the aforesaid multiple component grafts will be described in detail with respect to certain embodiments which comprise both cartilage-derived matrices and bone-derived matrices and are used for treating joints having osteochondral defects which involve both cartilage and bone tissue damage, the multiple component grafts are not limited to such embodiments. Rather, persons of ordinary skill will recognize that, depending on the types of tissue involved in defects to be treated, and the desired bioactivity to be provided at the tissue defect, the multiple component grafts may comprise combinations of matrices derived from any two or more types of tissue, such as, without limitation, adipose, amnion, artery, bone, cartilage, chorion, colon, dental, dermal, duodenal, endothelial, epithelial, fascial, gastrointestinal, growth plate, intervertebral disc, intestinal mucosa, intestinal serosa, ligament, liver, lung, mammary, meniscal, muscle, nerve, ovarian, parenchymal organ, pericardial, periosteal, peritoneal, placental (including amnion, chorion, amnionchorion, umbilical cord, and Whartons jelly), skin, spleen, stomach, synovial, tendon, testes, umbilical cord, urological, vascular, and vein.

The multiple component grafts described and contemplated herein may, for example, be used to treat defects in joints including, but not limited to, knees, hips, shoulders, temporomandibular joint (TMJ), elbows, ankles, and finger digits (e.g., knuckles). More particularly, multiple component grafts comprising a cartilage-derived matrix and a bone-derived matrix would be useful to treat osteochondral defects in such joints. The multiple component grafts described and contemplated herein may also be useful in treating other regions and structures of a subject, as may be deemed suitable by persons of ordinary skill in the relevant art. For example, the multiple component grafts described and contemplated herein may applied to more successfully treat tissue defects in areas or structures where tissues of different types exist adjacent or in contact with one another so that the defects involve more than one type of tissue. Defects in other regions and structures which could be beneficially treated with multiple component grafts described and contemplated herein include, without limitation, sternum, spine, ear, nose, etc. Additionally, defects in areas and structures which are hard to heal regions, such as where there is a transition from soft tissue types to hard tissue types, could be treated with the multiple component grafts. Such defects include those present in or proximate to joint entheses including, without limitation, fibrous entheses and fibrocartilagenous entheses such as those which exist at the juncture of ligament and bone and of tendon and bone.

An example of treating a naturally grading (transitioning) tissue such as entheses, could require attaching a compliant material (tendon), with a modulus on the order of 200 MPa, to a stiff material (bone), with a modulus on the order of 20 GPa, which presents a challenge (see, Lu, H. H., Thomopoulos, S., Functional attachment of soft tissues to bone: development, healing, and tissue engineering, Annu Rev Biomed Eng. 2013; 15:201-26; webpage: https://pubmed.ncbi.nlm.nih.gov/23642244/). A multiple component graft comprising sequential components which approximate or duplicate natural entheses (which may include various gradual changes in properties due to numerous natural factors such as interdigitation, fiber orientation etc., on a micrometer scale and spatial gradient in mineralization in nanometer scale) could be useful to treat various injuries and degeneration of intra-articular and extra articular entheses. These common anatomic sites of injuries may include intra-articular entheses such as the flexor tendon, the anterior cruciate ligament, the rotator cuff, and the meniscal root and extra-articular entheses may include the patellar tendon, the Achilles tendon, and the medial collateral ligament etc. (see, Derwin, K. A., et al., Enthesis repair Challenges and Opportunities for Effective Tendon-to-Bone Healing, J Bone Joint Surg Am. 2018 Aug. 15; 100(16): 1-7; webpage: https://pubmed.ncbi.nlm.nih.gov/30106830/).

In general, exemplary embodiments of multiple component grafts include those which comprise three components, including a first component comprising a muscle-derived matrix, a second component comprising an adipose-derived matrix and a third component comprising a dermis-derived matrix. Such a muscle-adipose-dermis multiple component graft could beneficially treat a deep wound such as where tendon and/or bone have been exposed, tunneling wounds, pressure ulcers, and third or fourth degree burns which may extend into subcutaneous tissue layers. Such deep wounds may, for example without limitation, extend through superficial and deep dermal tissue, through an underlying adipose tissue layer, and into underlying muscle tissue. A multiple component graft comprising a first component which includes a dermal tissue matrix, a second component including an adipose tissue matrix, and a third component including a muscle tissue matrix, may be implanted (or assembled prior to implanting, such that after implanting) each of the different first, second and third components to be proximate to and match the same type of tissue as present in each portion of the deep wound (tissue defect). For example, without limitation, the third component including a muscle tissue matrix is implanted proximate to the muscle portion in the deepest part of the wound, the second component including an adipose tissue matrix is implanted proximate an intermediate adipose portion of the wound, and the first component including a dermal tissue matrix is implanted proximate the most superficial dermal portion of the wound. In such embodiments, where the components are implanted sequentially to form the multiple component graft, the third muscle matrix component would be implanted first, followed by the second adipose matrix component, and the first dermal matrix component would be implanted last. As described above, the physical forms of each component and matrix thereof is not limited. For example, the different graft layers (i.e., components) could, for example, be mechanically reduced and sequentially reformed tissue layers or laminated processed tissue sheets (or combination thereof).

The combination of either a moldable bone “putty” or pre-shaped bone graft with a cartilage “putty” implant (also moldable or pre-shaped) should provide a solution that allows for the filling of non-uniform defects (e.g., involving more than one type of tissue, such as osteochondral defects) with a range of shapes and sizes as these defects appear in real world clinical situations, without the unnecessary removal of healthy joint tissues. Furthermore, when the tissue-derived matrices are in moldable form, such as “putty,” the multiple component graft is shapeable at the time of use and, therefore, can be shaped to fit into a defect to be treated. For example, without limitation, in subjects having a cartilage lesion with associated underlying subchondral bone defects, using a composite implant comprising both a bone-derived biomaterial and a cartilage-derived biomaterial at the defect site should allow the composite implant to recapitulate the microstructural and biologic architecture of a traditional osteochondral implant.

Osteochondral defects have both a bone portion and a cartilage portion, where the bone portion is generally located deeper in the defect, while the cartilage portion is generally closer to the surface. In accordance with the present invention, such a defect is filled with a bone-derived matrix (i.e., a first component) until the bone portion of the defect is appropriately filled. In some embodiments, autologous biologics, such as bone marrow, bone marrow concentrate, blood-derived products, PRP, PPP, or cells derived from the patient, may be added to the bone-derived matrix component. A cartilage-derived matrix (i.e., a second component) is then applied over the already-filled bone portion, until the cartilage portion of the defect is completely filled. In some embodiments, autologous biologics, such as bone marrow, bone marrow concentrate, blood-derived products, PRP, PPP, or cells derived from the patient, may also be added to the cartilage-derived matrix component. Furthermore, in some embodiments, the graft may include other additional materials which are capable of forming a covering over the implanted graft, such as without limitation, a periosteal patch, a coating of fibrin glue, etc., as selectable by persons of ordinary skill in the relevant art.

More particularly, an osteochondral joint defect is identified via imaging or direct visualization. The bone portion may be debrided/cleaned up and then filled with a bone-derived matrix. The bone-derived matrix is preferably an allograft bone matrix that can be formed or shaped to appropriately fill the bone portion of the defect. The bone-derived matrix may, for example, be composed of cortical bone fibers or particulates, cancellous bone chips or particulates, monolithic cortical bone, monolithic cancellous bone, demineralized, partially demineralized or non-demineralized, or a combination. The bone-derived matrix of a multiple component graft may, for example, be derived from cortical bone in the physical form of a sheet (monolithic, multi-piece, or molded or otherwise shaped from a plurality of fibers, granules or particles) which has been sized and shaped to match or otherwise conform to the osteochondral bone plate of the osteochondral joint defect to be treated.

Another exemplary embodiment of a multiple component graft in accordance with the description provided herein and which could be useful for more effectively treating an osteochondral defect could comprise three components including: a first component comprising a bone-derived matrix, a second component comprising a multiple tissue matrix which includes mixed bone and cartilage matrices, and a third component comprising a cartilage-derived matrix.

Such a multiple component graft would advantageously be formed by sequentially implanting the three components as follows: first implanting the first component comprising the bone-derived matrix proximate the bone portion of the osteochondral defect in an amount which completely or nearly completely fills the bone portion of the defect, then implanting the second component comprising the bone and cartilage (multiple) tissue matrix in an amount which results in the second component being in contact with both the bone and cartilage portions of the defect, and then implanting the third component comprising the cartilage-derived matrix proximate the cartilage portion of the defect in an amount sufficient to fill the remainder of the defect.

The type and form of the bone-derived matrix is not particularly limited and may be any known now or in the future to persons of ordinary skill in the relevant art. Many bone-derived matrices are now known including, without limitation, those described in U.S. Pat. Nos. 10,173,375, 10,130,736, 9,352,003, 8,883,210, 8,663,672 and U.S. Patent Application Publication Nos. 2016/0361171 and 20170266348, all of which are incorporated by reference herein. For example, without limitation, in some embodiments, the bone-derived matrix comprises cortical allograft demineralized bone particulate or fibers (fully or partially demineralized or not), cancellous allograft bone (chips, particulates, sponge, sheet, or solid core/shape, each being fully or partially demineralized, or not demineralized), viable bone matrix (e.g., cryopreserved, media-preserved or lyopreserved), or a combination thereof Such bone-derived matrices are typically derived from bone samples recovered from one or more donors and subjected to one or more processing steps including, but not limited to: size reduction by cutting, slicing, grating, grinding, milling, etc.; cleaning, soaking, and disinfecting by contacting with one or more solutions comprising water, buffered or unbuffered saline, sodium chloride, detergents, enzymes, peracetic acid, surfactants, antibiotics, alcohols, basal medium, etc.; partially or fully dehydrating by air-drying, heat drying, lyophilizing, chemical dehydration, etc.; and preserving by freezing, cryopreserving, lyophilizing, media-preserving with or without protectant agents (e.g., DMSO, glycerol, sugars, polyphenols, carotenoids, amino acids, etc.).

This configuration allows for irregular defects to be filled with the formable/moldable/shapeable bone-derived matrix. The bone-derived matrix may, for example, be a pre-shaped implant, such as cylinder or oval (whether a monolithic shape, or several monolithic pieces, or a molded shape comprising a plurality of fibers, particles, or combinations thereof) that can be placed in the defect after preparation of the bone portion of the defect using shaping instrumentation to match the implant shape and size. Similarly, a shaping instrument may be used to modify the shape and size of one or more of the tissue-derived matrices of the multiple component graft to fit into the defect to be treated. The bone-derived matrices components of multiple component grafts in accordance with the present invention may also include one or more additional materials such as: synthetic materials (e.g., ceramic, polymer, synthetic bone material, etc.), other exogenous materials (e.g., tri-calcium phosphate, hydroxyapatite, bioactive glass, porous metals such as titanium, tantalum, etc.), or extracellular matrix biomaterials, such as collagen, or natural- or animal-derived biomaterials, such as chitosan or alginate, or combinations thereof.

The type and form of the cartilage-derived matrix is not particularly limited and may be any known now or in the future to persons of ordinary skill in the relevant art. Several cartilage-derived matrices are now known including, without limitation, those described in U.S. Pat. Nos. 9,511,171, 8,292,968 and 8,883,210 and U.S. Patent Application Publication Nos. 2006/0210643, 2011/0196508 and 2017/0049930, all of which are incorporated herein by reference.

For example, the cartilage-derived matrix may be one or more of viable cartilage fibers (e.g., cartilage fibers containing viable native chondrocytes, cryopreserved, lyopreserved, media-preserved or not), lyophilized cartilage particles, or a combination thereof Cartilage-derived matrices may have any of several shapes and sizes including, without limitation, discs, ovals, sheets, putty, flowable fluid, slivers and other geometries, and may or may not contain viable cells. Such cartilage-derived matrices are typically derived from cartilage samples recovered from one or more donors and subjected to one or more processing steps including, but not limited to: size reduction by cutting, slicing, grating, grinding, milling, etc.; cleaning, soaking, and disinfecting by contacting with one or more solutions comprising water, buffered or unbuffered saline, sodium chloride, detergents, enzymes, peracetic acid, surfactants, antibiotics, alcohols, basal medium, etc.; partially or fully dehydrating by air-drying, heat drying, lyophilizing, chemical dehydration, etc.; and preserving by freezing, cryopreserving, lyophilizing, with or without protectant agents (e.g., DMSO, glycerol, sugars, polyphenols, carotenoids, amino acids, etc.).

In some embodiments, without limitation, the multiple component graft comprises a first component which includes a cartilage-derived matrix such as viable cartilage fibers, and a second component which includes a bone-derived matrix such as demineralized cortical bone fibers. In some embodiments, without limitation, the multiple component graft comprises a first component which includes a cartilage-derived matrix such as viable cartilage fibers, and a second component which includes a bone-derived matrix such as mineralized cortical-cancellous chips. In some embodiments, without limitation, the multiple component graft comprises a first component which includes a cartilage-derived matrix such as viable cartilage fibers, and a second component which includes a bone-derived matrix such as monolithic piece of cortical or cancellous or corticocancellous bone. In some embodiments, the multiple component graft comprises more than two components. In some embodiments, one or more of the components of the multiple component graft includes a combination (physically mixed or not, and homogenous or not) of two or more tissue-derived matrices, all of which are derived from the same type of tissue, but which have been processed differently or have different physical forms. For example, in an embodiment, a multiple component graft may comprise a component which comprises a bone-derived matrix of demineralized cortical fibers and another bone-derived matrix of mineralized viable cancellous chips. In another embodiment, a multiple component graft may comprise a component which comprises a cartilage-derived matrix of viable cartilage fibers and another cartilage-derived matrix of lyophilized cartilage particles.

The different components of the multiple component graft, as well as different tissue-derived matrices and multiple tissue matrices which may form each component, may be in the same or different physical form. As will be recognized by persons or ordinary skill in the relevant art, putty may include tissue fibers, granules, particles, or a combination thereof, with or without a biocompatible carrier. In some embodiments, the bone-derived matrix in the physical form of a putty is a settable putty which includes a settable or curable carrier (e.g., a reverse phase polymer or poloxamers, or a UV curable composition) to minimize subsidence of the bone-derived matrix. As will also be recognized by persons or ordinary skill in the relevant art, three-dimensional shapes such as discs, bricks, cylinders, etc., may be each be a monolithic piece of cut, sculpted, shaved or filed tissue, or molded (or otherwise shaped) pluralities of tissue fibers, granules, particles, or combinations thereof, with or without a biocompatible carrier, with or without some degree of drying. In some embodiments, where the multiple component graft comprises a cartilage-derived component and a bone-derived component, the cartilage-derived component is a cartilage-derived matrix in the physical form of putty and the bone-derived component is a bone-derived matrix in the physical form of a disc or putty. In other embodiments, the cartilage-derived component is a cartilage-derived matrix in the physical form of a disc and the bone-derived component is a bone-derived matrix in the physical form of putty or a disc. In still other embodiments, the bone-derived component may comprise a first bone-derived matrix in the physical form of a putty and a second bone-derived matrix in the physical form of a disc, and the cartilage-derived component may comprise a cartilage-derived matrix in the physical form of either putty or a disc.

In some embodiments, one or more of the components of the multiple component graft is a multiple tissue matrix which includes a combination (physically mixed or not, and homogenous or not) of two or more tissue-derived matrices which are derived from different types of tissue. For example, a multiple component graft may comprise a first component (for filling the bone portion of an osteochondral defect) which comprises a physical mixture of a bone-derived matrix of demineralized cortical fibers and a cartilage-derived matrix of lyophilized cartilage particles, and a second component (for applying over the first component to fill the cartilage portion of the defect) which comprises a cartilage-derived matrix of viable cartilage fibers.

In some embodiments, the multiple component graft comprises at least two components, wherein a first component comprises a bone-derived matrix and a second component comprises a cartilage-derived matrix which is in the physical form of lyophilized non-viable cartilage particles (cartilage allograft matrix, or “CAM”). In such embodiments, the cartilage-derived matrix (CAM) is applied or implanted as a top layer of the graft, i.e., at or close to the surface of the osteochondral defect being treated, with or without an adhesive (such as fibrin glue or similar additional material) to provide a retaining layer, or a stabilizing or protective barrier to restrict or minimize transfer of air and fluid into and out of the osteochondral defect and multiple component graft therein.

As still another example, a multiple tissue matrix may comprise two or more pieces of non-demineralized cancellous bone which have been cleaned, cut and milled or otherwise shaped into a 3-D shape (e.g., cylinders), which are assembled with one another (e.g., aligned and held together with a bone pin, or other fixation device or material), with a plurality of separately prepared cartilage particles which have been added to one or more of the cancellous bone pieces such that the cartilage particles infiltrate the cancellous bone pieces (since they are somewhat porous) at least to some degree, and may be lyophilized together or not. An exemplary embodiment of a multiple component graft may comprise a first component which includes such a multiple tissue matrix comprising cancellous bone pieces assembled together and infiltrated with cartilage particles, as well as a second component which includes viable cartilage particles, where the first component would be implanted proximate to the bone portion of the defect (with or without rehydration by addition of a biocompatible fluid) and the second component would be implanted proximate the cartilage portion of the defect.

In some embodiments, the multiple component graft may further comprise an additional material capable of forming a barrier (i.e., a “barrier material”) and which is applied or implanted (directly or indirectly) over at least one of the tissue-derived matrices of the multiple component graft. The barrier material provides a barrier to restrict or minimize transfer of air and fluid into and out of the osteochondral defect and multiple component graft therein. Such barrier material may, for example without limitation, comprise one or more of: a glue, an adhesive, a sealant, a coating, a curable substance, and combinations thereof. More specifically, exemplary barrier materials include, without limitation, cyanoacrylates, epoxies, silicones, fibrin glue, epoxy-polyurethane blends, UV/LED curing systems, acrylate resins, and combinations thereof.

In some embodiments, the multiple component graft comprises three or more components. For example, without limitation, the multiple component graft may comprise a first component comprising a bone-derived matrix, and a second component comprising a cartilage-derived matrix, and a third component comprising a placenta-derived matrix. In such embodiments, any one or more of the first, second and third components may be a tissue-derived matrix or a multiple tissue matrix. For example, without limitation, the first component may comprise two or more types of bone-derived matrices, such as a cortical bone-derived matrix and a cancellous bone-derived matrix, as described above in other exemplary embodiments. In some embodiments, the third component may comprise two or more types of placenta-derived matrices, such as two or more of an amnion-derived matrix, a chorion-derived matrix, and an umbilical cord-derived matrix. Additionally, in such embodiments, each of the tissue-derived matrices or multiple tissue matrices comprising the first, second, and third components, has at least one physical form which may be the same or different physical form as the other matrices. Furthermore, any one or more of the first, second and third components may be viable or not. Any one or more of the first, second and third components may include one or more additional materials.

Additionally, as will be familiar to persons of ordinary skill in the relevant art, fixation methods may be used to secure the multiple component graft, for example, to secure one or more individual tissue-derived matrices or multiple tissue matrices as each is applied or implanted, or to be applied after all components have been assembled, applied or implanted in a defect, or both. Fixation methods are not particularly, limited and may include physical methods, such as pins, screws, or a covering membrane, or chemical fixation such as glues (for example, fibrin glue, acrylates, etc.). Such fixation methods may be applied to any one or more of the two or more components of a multiple component graft, or any one or more of the constituent matrices of one or more of the components, as deemed appropriate, desired and effective by persons ordinary skill in the relevant art. Such fixation methods may be used to affix, adhere, or connect one or more components and matrices to one another, to tissues of the tissue defect, or both.

In some embodiments, the multiple component graft comprises a first component which comprises a first tissue-derived matrix or a first multiple tissue matrix which has a physical form selected from pieces, particles, fibers, powder, sheets, putty, flowable fluid, and combinations thereof, and a second component which comprises a second tissue-derived matrix having a three-dimensional physical form which includes one or more matrix receiving features including, without limitation, openings, recesses, cavities, surfaces, troughs, lumens, or combinations thereof, for receiving at least a portion of the first component. In an exemplary embodiment, the first tissue-derived matrix or first multiple tissue matrix includes a first bone-derived matrix which may be viable and has a physical form selected from particulate, powder, granule, putty, flowable fluid, and combinations thereof, and the second tissue-derived matrix includes a second bone-derived matrix which is in monolithic or multi-piece physical form and has one or more matrix receiving features including, without limitation, openings, recesses, cavities, surfaces, troughs, lumens, or combinations thereof, for receiving at least a portion of the first bone-derived matrix therein or thereon. In such an exemplary embodiment, at least a portion of the first bone-derived matrix is applied, glued, layered, inserted, or otherwise placed into or onto at least one of the matrix receiving features. For instance, the second bone-derived matrix may be a monolithic cortical bone piece which is sized and shaped for use a spinal spacer in spine reconstruction procedures and has one or more recesses, openings, or lumens, and the first bone-derived matrix may be viable or nonviable bone granules which fill at least one of the recesses or openings of the cortical spinal spacer. As another example, the second bone-derived matrix may be a multi-piece bone implant capable of providing structural and weight-bearing support when used for bone reconstruction and having one or more recesses, openings, or lumens, recesses, openings, or lumens, and the first bone-derived matrix may be viable or nonviable bone powder in a flowable which fill at least one of the recesses, openings, or lumens of the multi-piece bone implant. As still another example, the first bone-derived matrix may have monolithic or multi-piece physical form with at least one surface and the first bone-derived matrix may have a flowable fluid physical form (e.g., powder or granules mixed with a biocompatible liquid or gel carrier comprising an adhesive) which is applied to or layered on at least a portion of the surface of the monolithic or multi-piece bone-derived matrix to form a layer or coating.

In an exemplary embodiment, a multiple component graft may comprise at least a first component which comprises a cancellous bone matrix and a second component which comprises a cortical bone matrix which is assembled in contact with the first component, either before or during implanting proximate to a tissue defect which includes at least a portion of subchondral bone and wherein the assembled multiple component graft is sized and shaped to be similar or conform to the architecture (size and shape) of the portion of subchondral bone to be treated in the defect. For example, without limitation, the second component comprising a cortical bone-derived matrix might be a shell or cover element layered on top of the first component comprising a cancellous-derived matrix.

The sequential steps of applying the bone-derived matrix and applying the cartilage-derived matrix may be performed during the same clinical procedure or during separate procedures. In other words, the bone portion of the osteochondral defect may be filled during one procedure, wait for the bone portion to remodel (e.g., regenerate) into de novo bone tissue, and then, in a subsequent procedure, the cartilage-derived matrix may be applied to fill the cartilage portion of the osteochondral defect. In some embodiments in which one or more components of a multiple component graft are implanted during separate procedures, i.e., the procedures and implantations may be separated by periods of time extending from hours to days, weeks, months, or even a year or more, depending on the nature of the defect being treated and the progress of healing at the defect site, as determinable by persons of ordinary skill in the relevant art. Thus, methods of producing or using the multiple component grafts may, in some embodiments, include sequentially implanting each component proximate to or in contact with a portion of a tissue defect or wound, where each component may be implanted hours, days, weeks, months, or even a year or more before or after other components.

In some embodiments, the multiple component grafts may include one or more movement facilitation features such as, without limitation, one or more passages, perforations, conduits, channels, or combinations thereof, which enable, facilitate, increase (or combinations thereof) the movement (e.g., infiltration, migration, flow, penetration, etc.) of one or more materials into, out of, and through at least a portion of the graft. Each of the movement facilitation features may, independently of one another, extend partially or completely into or through the multiple component graft or portions thereof. Furthermore, each of the movement facilitation features may, independently of one another, be linear or not, continuous, interrupted, intermittent, segmented, and any other geometry or arrangement desired. The movement facilitation features may be produced or formed in the multiple component graft or portions thereof by any of several methods as known and determinable by persons of ordinary skill in the relevant art such as, for example without limitation, drilling, laser ablation, during or using 3-D printing, and combinations thereof.

The materials whose movement is enabled, facilitated, increased, etc., by the movement facilitation features include, without limitation, one or more of: cells (endogenous, exogenous, etc.), growth factors, blood and derivatives thereof (e.g., PRP, PPP, etc.), bone marrow, other biological fluids, biocompatible fluids, antibiotics, other therapeutic substances. The movement of such materials may, for example without limitation, be between one or more of: the tissues of and near the tissue defect being treated, the multiple component graft, one or more components of the graft, portions of the components, etc. For instance, the patient's (subject's) cells, growth factors, PRP, PPP, biological fluid, or a combination thereof, may migrate from the patient and infiltrate one or more components of the graft. Similarly, cells, growth factors, therapeutic substances, or a combination thereof, may migrate, pass or otherwise move from the multiple component graft to tissues of the defect or other tissues of the patient, or from one component, matrix, or portion of the graft to another.

For example, in some embodiments where a three-component muscle-adipose-dermis multiple component graft is used to treat a deep wound, perforations or channels may be introduced or formed throughout the multiple component graft or through only one or more components or portions thereof. In some embodiments, the movement of material may be inwards, from the subject's surrounding tissues or external to the graft and wound or defect, then through the graft or at least portions thereof, and toward the wound bed (i.e., toward the damaged tissue of the defect, and the juncture of the damaged and healthy tissues), to enhance infiltration and incorporation of cells, growth factors, cytokines, anti-inflammatory agents, anti-microbial agents, and combinations thereof, into the tissue defect. The movement of materials through movement facilitation features may, also or alternatively, be outwards, from the tissue defect and towards the surface of the graft for egress or removal of wound fluid, infection, excess materials, etc.

Other biologics, therapeutic substances, or drugs may be added to the implant prior, during or after implantation. These include antibiotics, antimicrobial, anti-inflammatory, growth factors, proteins, hormones, etc.

The implant may be stored at room temperature or preserved at sub-zero temperatures to preserve the viability of the cells present, if any, in the tissue-derived matrices.

The components of the graft (i.e., each of the tissue-derived matrices) may each be provided independently of the others for maximum flexibility, or they may be provided separately from one another, but collected and provided together in a kit for convenience. Instrumentation (e.g., delivery device(s)) may be multiple use, single use, or a combination thereof, and also provided independently or in a kit, preloaded or not, with the tissue-derived matrix components.

It will be understood that the embodiments of the present invention described hereinabove are merely exemplary and that a person skilled in the art may make variations and modifications without departing from the spirit and scope of the invention. All such variations and modifications are intended to be included within the scope of the present invention.

### EXAMPLES

Multiple component osteochondral grafts in accordance with the invention described herein are created in situ in the stifle joint of Sinclair mini-pigs. The stifle joint is the quadruped equivalent of the human knee, and this a clinically relevant model for assessing cartilage defect healing. All animals (tests subjects) are prepared for arthrotomies of the right or left stifle joints, and each defect is located on the anterior face of the medial condyle, proximal to the patellar notch.

For each animal, a full-thickness defect is created, including both the cartilage and cortical layers, to fresh bleeding bone. The defect diameter does not exceed 8.5 mm and is not less than 7.5 mm. Margins are inspected, and edges cleaned. Finally, the wound is thoroughly irrigated and bone fragments removed. The osteochondral implant is then created in situ by placing cortico/cancellous granules into the defect site to a depth matching the thickness of the bone layer in the defect. After this, a cartilage-derived matrix comprising a 50/50 mixture of viable cartilage fibers and lyophilized cartilage particles is placed to a depth consistent with the outer layer of the cartilage. (Alternatively, based on the judgment of the medical practitioner performing the procedure, the cartilage-derived matrix may comprise 100% viable cartilage fibers, or 100% lyophilized cartilage particles.)

The patella is then returned to the notch, and the capsule is closed using interrupted absorbable sutures. The skin is closed with subcuticular interrupted sutures and surgical glue is, optionally, applied. Healing of the cartilage defect will be assessed at 3 and 6 months.

Observable repair and/or regeneration of both bone and cartilage proximate the defect is expected at each of 3 and 6 months.

We claim:

1. A multiple component graft for treating a tissue defect, comprising two or more components, each of which comprises a tissue-derived matrix or a multiple tissue matrix, wherein at least two of the components are derived from and comprise different types of tissue.

2. The multiple component graft of claim 1, wherein the types of tissue are selected from: adipose, amnion, amniochorion, artery, bone, cartilage, chorion, colon, dental, dermal, duodenal, endothelial, epithelial, fascial, gastrointestinal, growth plate, intervertebral disc, intestinal mucosa, intestinal serosa, ligament, liver, lung, mammary, meniscal, muscle, nerve, ovarian, parenchymal organ, pericardial, periosteal, peritoneal, skin, spleen, stomach, synovial, tendon, testes, umbilical cord, urological, vascular, vein, Wharton jelly, and combinations thereof.

3. The multiple component graft of claim 1, wherein each tissue-derived matrix and multiple tissue matrix, independently, has a physical form selected from: pieces, particles, fibers, powder, sheets, putty, flowable fluid, three-dimensional shapes, and combinations thereof.

4. The multiple component graft of claim 3, wherein the three-dimensional shapes are selected from: monolithic, multi-piece, shapes otherwise formed, molded or shaped from other physical forms, and combinations thereof.

5. The multiple component graft of claim 1, comprising a first component which comprises a first tissue-derived matrix or a first multiple tissue matrix which includes at least a first type of tissue and at least one physical form, and a second component which comprises a second tissue-derived matrix or a second multiple tissue matrix which includes at least a second type of tissue which is different from the first type of tissue and at least one physical form.

6. The multiple component graft of claim 5, wherein the first component comprises a bone-derived matrix, the first type of tissue is bone, and the first physical form is selected from pieces, granules, particles, powder, fibers, putty, and a combination thereof, and the second component comprises a cartilage-derived matrix, the second type of tissue is cartilage, and the second physical form is selected from pieces, granules, particles, powder, fibers, putty, and a combination thereof.

7. The multiple component graft of claim 1, further comprising one or more additional materials

8. The multiple component graft of claim 7, wherein each of the one or more additional materials is combined or contacted with one or more of the two or more components before, during or after implanting each of the two or more components proximate the tissue defect.

9. The multiple component graft of claim 5, further comprising a third component which comprises a third tissue-derived matrix or a third multiple tissue matrix which includes at least a third type of tissue, which is different from the first and second types of tissue, and at least one physical form.

10. The multiple component graft of claim 9, wherein the first component comprises a bone-derived matrix and the first type of tissue is bone, the second component comprises a cartilage-derived matrix and the second type of tissue is cartilage, the third component comprises an amnion-derived matrix and the third type of tissue is amnion, and the at least one physical form of each of the bone-derived matrix, cartilage-derived matrix and amnion-derived matrix is, independently, selected from pieces, granules, particles, powder, fibers, putty, and a combination thereof.

11. The multiple component graft of claim 9, further comprising one or more additional materials

12. The multiple component graft of claim 11, wherein each of the one or more additional materials is combined or contacted with one or more of the two or more components before, during or after implanting each of the two or more components proximate the tissue defect.

13. A method of producing the multiple component graft of claim 1, comprising the steps of:
providing the two or more components, and
sequentially implanting each of the two or more components, one at a time, proximate the tissue defect.

14. The method of claim 13, wherein the step of providing the two or more components is performed by obtaining: a first tissue-derived matrix or a first multiple tissue matrix which includes at least a first type of tissue and at least one physical form, and a second component which comprises a second tissue-derived matrix or a second multiple tissue matrix which includes at least a second type of tissue which is different from the first type of tissue and at least one physical form, and wherein the step of sequentially implanting is performed by implanting each of the two or more components, one at a time, proximate the tissue defect, wherein either the of the first or second components is implanted first.

15. The method of claim 14, wherein the tissue defect is an osteochondral defect in a joint having a bone portion and a cartilage portion, where the bone portion is located deeper in the defect, wherein the first component of the multiple component graft comprises a bone-derived matrix and the first type of tissue is bone, the second component of the multiple component graft comprises a cartilage-derived matrix and the second type of tissue is cartilage, the at least one physical form of each of the bone-derived matrix and cartilage-derived matrix is, independently, selected from pieces, granules, particles, powder, fibers, putty, and a combination thereof, and wherein the step of sequentially implanting is performed by first implanting the bone-derived component proximate the bone portion of the osteochondral defect, and thereafter, implanting the cartilage-derived matrix proximate the bone-derived matrix and the cartilage portion of the osteochondral defect.

16. The method of claim 14, wherein the tissue defect is an osteochondral defect in a joint having a bone portion and a cartilage portion, where the bone portion is located deeper in the defect, wherein the first component of the multiple component graft is a multiple tissue matrix which comprises a mixture of lyophilized cancellous bone granules and cortical bone fibers, and the second component of the multiple component graft comprises a cartilage-derived matrix, the second type of tissue is cartilage, and the at least one physical form of the cartilage-derived matrix is selected from pieces, granules, particles, powder, fibers, putty, and a combination thereof, and wherein the step of sequentially implanting is performed by first implanting the first component proximate the bone portion of the osteochondral defect, and thereafter, implanting the cartilage-derived matrix proximate the first component and the cartilage portion of the osteochondral defect.

17. The method of claim 13, wherein the multiple component graft further comprises one or more additional materials, and the method further comprises the step of combining or contacting each of the one or more additional materials with at least one of the two or more components of the multiple component graft before, during or after sequentially implanting each of the two or more components.

18. A method of producing the multiple component graft of claim 1, comprising the steps of:
providing the two or more components, and
prior to implanting the two or more components, combining two or more of the two or more components together to form one or more composite components and optionally, leaving one or more of the two or more components as one or more uncombined components, and
sequentially implanting each of the one or more composite components and, if present, each of the one or more uncombined components, one at a time, proximate the tissue defect.

19. A method for treating an external or internal tissue defect of a subject using the multiple component graft of claim 1, comprising the steps of:
optionally, preparing the tissue defect to receive the multiple component graft;
providing the multiple component graft; and
implanting the multiple component graft proximate the tissue defect according to the method of claim 13.

20. A method for treating an external or internal tissue defect of a subject using the multiple component graft of claim 1, comprising the steps of:
optionally, preparing the tissue defect to receive the multiple component graft;
providing the multiple component graft; and
implanting the multiple component graft proximate the tissue defect according to the method of claim 18.
